(19) United States
(12) Patent Application Publication
(10) Pub. No.: US 2011/0208244 A1
(43) Pub. Date: Aug. 25, 2011

(54) INTERSPINOUS SUPPORT AND METHOD FOR FIXING SAME

(76) Inventor: Jae Hyuk Shin, Seoul (KR)

(21) Appl. No.: 13/127,202

(22) PCT Filed: Nov. 5, 2009
(86) PCT No.: PCT/KR2009/006481

§ 371 Date: May 2, 2011

(30) Foreign Application Priority Data
Nov. 7, 2008 (KR) 10-2008-0110593

(51) Int. Cl. A61B 17/70; A61B 17/88

(52) U.S. Cl. 606/249; 606/279

(57) ABSTRACT

Disclosed herein is a support between spinous processes and method for fixing the same when a surgery on a backbone is unavoidable due to spinal stenosis or other lumbar lesions, wherein the support 100 between spinous processes of the present invention is comprised of a main body 110 made of a butterfly-shaped support part 111 with rings at both sides thereof and receiving parts 112 formed between the both wings; two rings 120 formed at one side of the main body 110; and two straps 130 whose one ends are attached to the other side of the main body 110 and the other ends are attached to two half-moon-shaped needles 140.

### TECHNICAL FIELD

The present invention relates to a support between spinous processes and method for fixing the same when a surgery on a backbone is unavoidable due to spinal stenosis or other lumbar lesions, and more particularly, to a support between spinous processes and method for fixing the same for maintaining a uniform interval between spinous processes of backbones and for allowing segmental movement of the backbones by inserting and securing the support between spinous processes on adjacent backbones.

### BACKGROUND ART

As shown in FIG. 1, a backbone 10 of a human body includes a robust vertebral bodies 11 functioning as a pillar; an intervertebral disc 14 between the vertebral bodies 11; spinous processes 13 on the back of the vertebral bodies; and spinal canals 12 between the vertebral bodies 11 and the spinous processes 13 through which vertebral nerves pass.

One of the main causes of spinal lesions is that a degenerative change resulting from aging of a human body narrows the area of a spinal canal to thereby increase the pressure on a nerve passing therethrough, and that the segmented spine loses stability and adds instability due to the degenerative change of joints.

The causes of a narrowed spinal canal include a thickened ligamentum flavum in the spinal canal due to the degeneration, protrusion of an intervertebral disc which effectuate the narrowing of the spinal canal, and a reduced distance between an upper and a lower backbones due to degenerative change which results in in the loss of elasticity of the intervertebral disc, and degeneration of a bone in shape due to bony degeneration of the backbone itself which encroaches the area of the spinal canal, for example.

The cause of the instability is degeneration of the intervertebral disc, which is an adjacent joint of two backbones, and a loosened connection between both-side back joints due to degeneration thereof, accompanied by weakened muscle ligaments in consequence, etc.

Accordingly, the main stream approaches of treating a spinal lesion are directed to relieving a pressure on the nerve to achieve relevant stability, and a classical method among them is to relieve a pressure on the backbone and then to agglutinate the bone. However, recently, a treatment called “dynamic fixation”, which preserves the movability of the backbone segment even after the treatment, as opposed to the notion of fixing a movable backbone, has come into spotlight.

An instrument insertion treatment, one of the dynamic fixation methodologies, inserts a support between the spinous processes 13 of a backbone, which may prevent lumbago caused by an excessive lumbar lordosis.

Based on the currently available agglutination treatment, after the medical surgery, movability of the backbone segment is lost, the movable range of the backbone lordosis is restricted, and the chances of degeneration of an adjacent backbone segment are increased. However, since the dynamic fixation permits segmental movement of the operated part, the treatment is characterized by a smooth organic function and less likelihood of a degeneration of adjacent segments.

The dynamic fixation methodologies include the Gref fixation, Dynesys fixation, memory loop treatment, and bio-flex fixation.

These days, however, DIAM (Device for Intervertebral Assisted Motion) is widely used as an option among those methodologies.

DIAM uses a structure of a butterfly-shaped silicon instrument with rings at both sides thereof (as shown in FIG. 2) and a dangled silicon strap with a needle. The silicon strap is wound around the upper and lower spinous processes and is fixed to the main body. During the surgery, the skin is incised about 3-cm long and the DIAM is inserted into the space between bones at the vertebral back (spinous processes) and secured by winding it to bones of spinous processes at both sides with a lace using, depending on the situation, high-tech equipments such as a surgical loupe or surgical microscope.

Thus, unlike the traditional backbone agglutination methodology that fixes the spinous processes with a screw, DIAM permits preservation of the movability of the backbones even after the surgery, and helps to reduce damages to muscles and surrounding tissues by minimizing the peeled area.

Additionally, DIAM has advantages of less bleeding during the surgery than backbone agglutination methodology, less scars and pains after the surgery, and fast recovery of the patient enough to allow the patient to walk the next day after the surgery is done.

Korean Registered Patent Nos. 10-0620114, 0620115, 0701575, 0701573, and 0701574 are examples of the insertion material between backbones used in such surgeries.

A spinal pillar insertion material according to the disclosed inventions includes a spacer having two opposite notches that receive spinous processes of backbones, and a strap for fixing two backbones to the spacer, wherein the spacer contains a through hole penetrating the two opposite notches for the strap to pass therethrough, and the strap fixes two backbones to the spacer by penetrating the through hole and binding the spacer and the two spinous processes in a shape of figure eight.

However, according to the above-mentioned inventions, fixing the insertion material for spine between spinous processes of two backbones needs complicated surgical procedures such as passing the strap into the through hole and binding the spacer and two spinous processes in a shape of figure eight. This necessitates peeling of left and right muscles on the spinous processes.

Therefore, it would be very convenient and efficient in various aspects if DIAM can be performed while muscles at only one side are peeled over the spinous processes 13, which may result in a smaller surgical area, less physical burden to the patient, and thus much simpler surgical procedures.

### DETAILED DESCRIPTION

### Technical Problems

It is thus an object of the present invention to provide a support between spinous processes and a method for fixing the same, which modifies the structure of a currently available DIAM in order to perform a surgery without the need of peeling muscles on both sides of the spinous processes 13 but only peeling muscles on only one side.

### Technical Solutions

To solve the objective, the present invention provides a structure a main body comprising a support part having a size to be inserted between spinous processes of backbones and receiving parts accommodating the spinous processes therein with protruded top and bottom sides of the support part; two rack holes formed at one side of the main body; and two straps attached to an other side of the main body.

### Advantageous Effects

Unlike currently available vertebral inserting material, a surgery can be performed with only single-side muscles of lumbar muscle peeled off, so that physical burden to the patient will be relieved, the surgery will be simpler with a shorter operation time, and thus the time for recovery will be significantly reduced.

### BRIEF DESCRIPTION OF DRAWINGS

FIG. 1 is a perspective view of a human body backbone;

FIG. 2 is a front view of a currently available inter-backbone inserting material;

FIG. 3 is a perspective view of a support between spinous processes according to the present invention;

FIGS. 5 through 9 are diagrams for describing a method for fixing a support between spinous processes according to the present invention; and

FIGS. 10 through 11 are diagrams for describing another embodiment of a method for fixing a support between spinous processes according to the present invention.

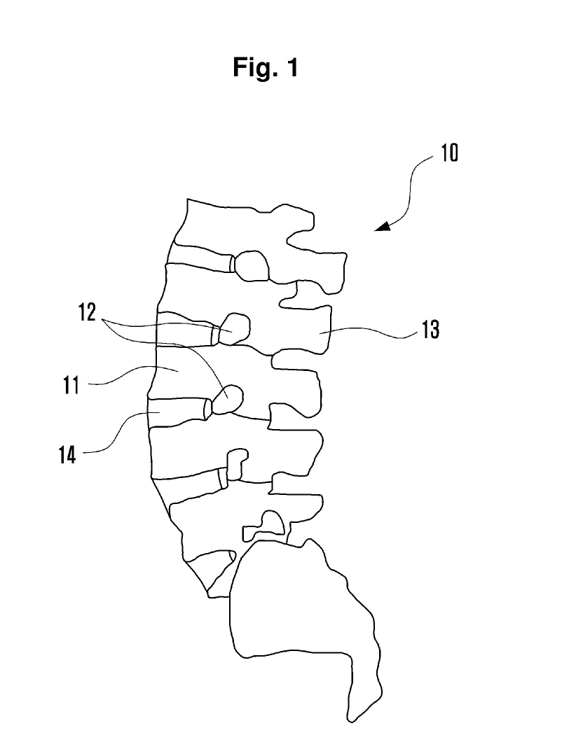

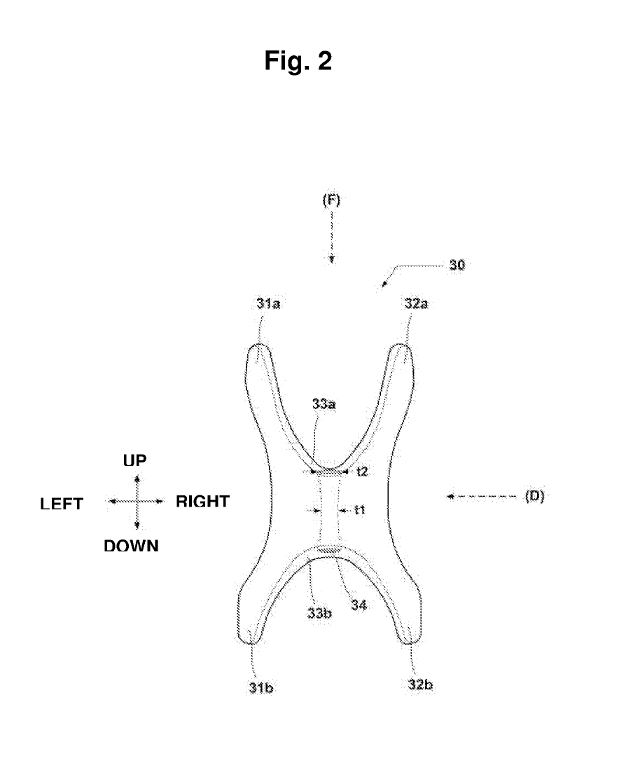

### BEST MODES FOR PRACTICING INVENTION

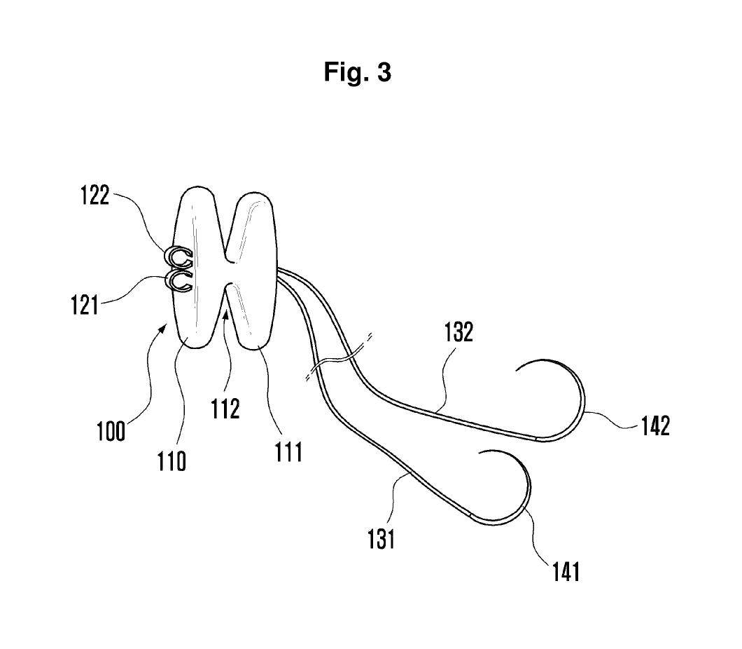

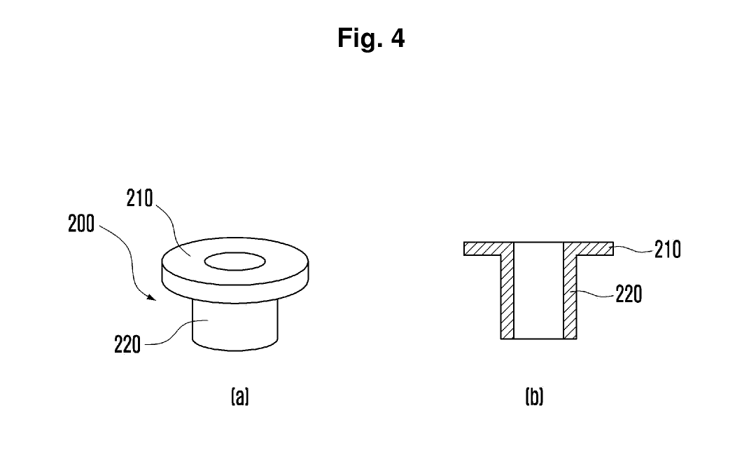

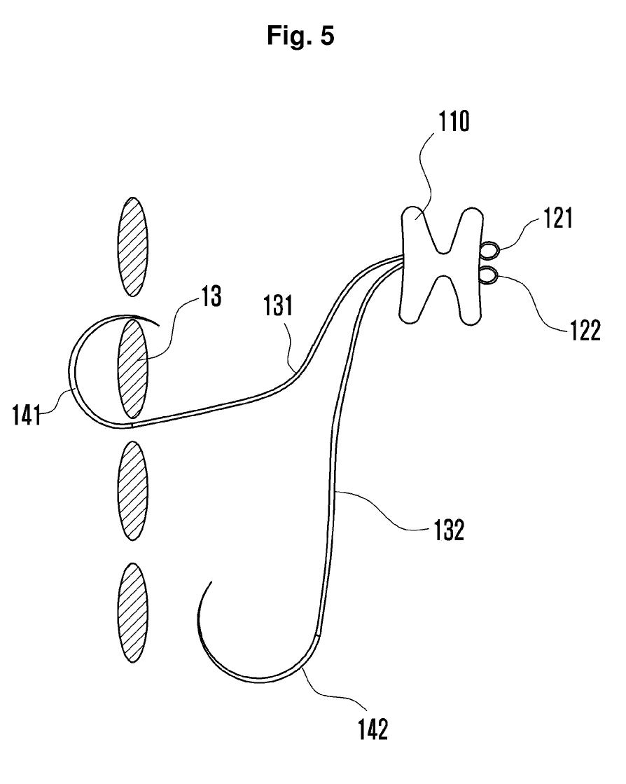

FIG. 3 is a perspective view of a support between spinous processes according to the invention, and FIG. 4 is perspective and cross-sectional views of a strap fixing pin. FIGS. 5 through 9 are diagrams illustrating a fixing method of the support between spinous processes according to the present invention.

Hereinafter, the present invention will be described in detail with reference to the accompanying drawings.

As shown in FIG. 3, a support 100 between spinous processes according to the present invention comprises a butterfly-shaped main body 110; two rings 120, 121 formed at one side of the main body 110; and straps 131, 132 whose one ends are attached to the other side of the main body 110 and the other ends are attached to two half-moon-shaped needles 141, 142.

A main body 110 comprises wing-shaped support parts 111 and receiving parts 112 between the wing-shaped support parts 111.

The main body 110 is made of elastic silicon material, and covered with polyester cloth harmless to a human body.

The reason that the main body 110 is made of elastic silicon material is to allow it to serve as a bumper that elastically supports the backbone part through the two spinous processes 13 and also to allow a segmental movement of the spinous processes 13.

The needle 142 attached to the strap 140 is of a half-moon shape, and a warping angle is designed to be over 180° following a circle. For a smooth surgery, the warping angle of the needle 140 should be designed to exceed 180° following a circle. The reason will be explained later.

Therefore, the support between the spinous processes according to the present invention is characterized by:

a main body 110 comprising a support part 111 with a shape of bilateral wings of a butterfly and receiving parts 112 each formed between the bilateral wings of the support part 111;

two rings 121, 122 formed at one side of the main body 110; and

two straps 131, 132 to which half-moon shaped needles 141, 142 are attached, the two straps 131, 132 being attached to the other side of the main body 110.

On the one hand, the main body 110 is characterized in that the main body 110 is made of elastic silicon material, covered with polyester cloth harmless to a human body.

Also, the needles 141, 142 are characterized in that a warp angle need to be over 180° following a circle.

Hereinafter, a method of fixing interspinous process support to spinous processes 13 according to the present invention will be described.

First, the interspinous process support and a strap fixing pin 200 according to the present invention are prepared. A strap fixing pin 200 is made of metal as shown in FIG. 4, comprised of a body part 220 of a flange 210, which is available from medical device merchandisers.

Here, the pressure on the nerve can be relieved by peeling back muscles at only one side on the spinous processes 13 with respect to the spinous processes 13 to be operated, then removing the elements that have encroached the area inside the spinal canal by surgery, e.g., by removing thickened ligamentum flavum and a protruded intervertebral disc.

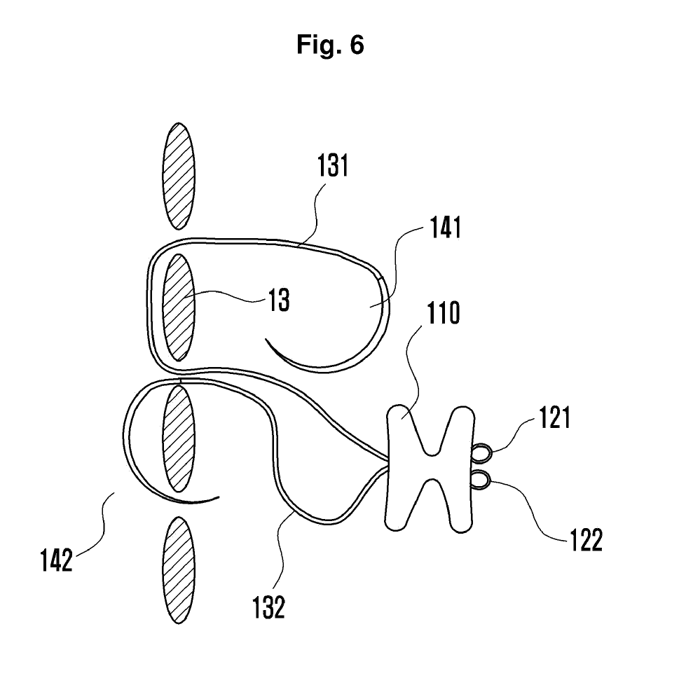

Thereafter, as shown in FIG. 5, the needle 141 of the upper strap 131 attached to the interspinous process support 100 is inserted from the peeled side of the back muscle between the two upper and lower spinous processes 13, to pass around the upper spinous process 13, and then the end of the needle 141 is pulled with a forceps. Afterwards, as illustrated in FIG. 6, the needle 142 of the lower part strap 132 is also inserted between the two upper and lower spinous processes 13, to pass around the lower part spinous process 13, and then the end of the needle 142 is pulled with another forceps.

At this time, the needle 141 has to enter between the two upper and lower spinous processes 13 and pass around the upper spinous process 13, but if a warp angle of needles 141, 142 is below 180° following a circle, the needles 141, 142 which have entered from one side of the spinous process 13, which is a straight line when one side of the backbone muscles are peeled, may not pass around the spinous process 13.

This is because, assuming that a surgeon holds the base portion of the needle at one side where the needle enters, when a needle all the way pass around the spinous process, the head of the needle is still located at the opposite side with respect to right and left sides of the spinous processes.

In this case, for the needle to pass around the spinous process 13, it is necessary that a forceps or pincette is inserted from a peeled side to an unpeeled opposite side from where the needle came out, then by stirring up between muscles and soft tissues to find the location of the head of the needle, which may result in additional damages to soft tissues.

However, if the warp angle of the needle 140 is over 180° following a circle, the head of the needle inserted at the peeled side passes through the unpeeled part and returns through the spinous processes 13.

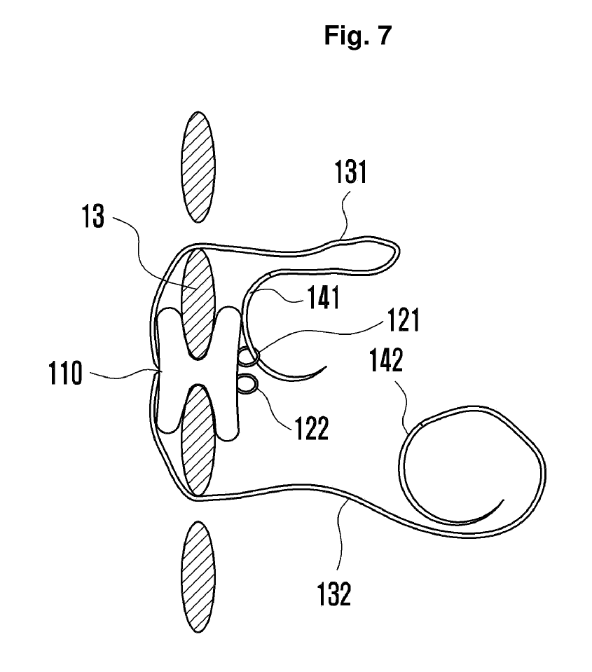

Thereafter, as shown in FIG. 7, the interspinous process support 100 is inserted between two upper and lower spinous processes 13 using a dedicated insertion device, the needle 141 is passed through to the upper ring 121 and pulled so that the upper strap 131 passes through the upper ring 121, and also, the lower strap 132 passes through the lower part ring 122 in the same manner.

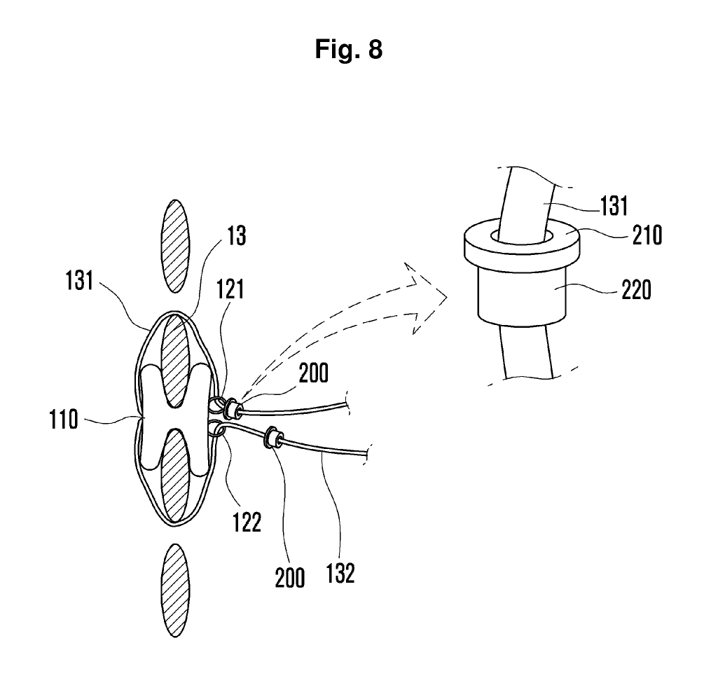

Thereafter, as shown in FIG. 8, the upper strap 131 is tightly pulled and a strap fixing pin 200 is inserted from where the needle 141 is located. When the body 220 is pressed with a plier with the flange 210 of the strap fixing pin 200 facing the upper ring 121, the upper strap 131 is prevented from being unwrapped by the strap fixing pin 200. Here, the flange 210 serves as a stopper.

Likewise, the lower part strap 132 is fixed by a strap fixing pin 200 in the same manner.

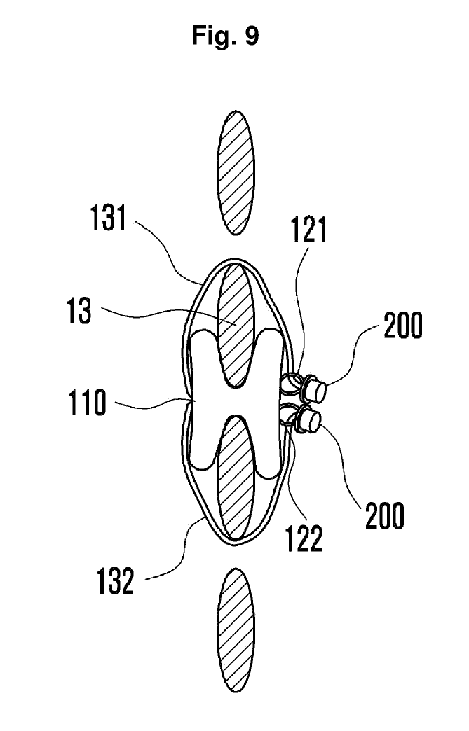

Thereafter, the straps 131, 132 exposed outside the lower part of the body 220 of the strap fixing pin 200 are cut, as shown in FIG. 9, and the interspinous process support 100 between spinous processes 13 is strained and fixed by straps 131, 132.

Therefore, the present invention provides a method for fixing the interspinous process support including:

a step of preparing an interspinous process support and a strap fixing pin according to the invention;

a step of peeling back muscles at only one side on the spinous processes 13 with respect to the spinous processes 13 to be operated, then removing the elements that have encroached the area inside the spinal canal by surgery;

a step of inserting the needle of the upper strap attached to the interspinous process support from the peeled side of the back muscle between the two upper and lower spinous processes, to pass around the upper spinous process and then pulling the end of the needle with a forceps;

a step of inserting the needle of the lower part strap between the two upper and lower spinous processes, to pass around the lower part spinous process, and then pulling the end of the needle with another forceps;

a step of inserting the interspinous support between the two upper and lower spinous processes to be safely received between the spinous processes;

a step of passing each of upper and lower straps through upper and lower rings, respectively, passing it through a strap fixing pin, and fixing the upper and lower straps to the interspinous process support;

a step of cutting a strap exposed outside the strap fixing pin; and

a step of sealing the peeled parts.

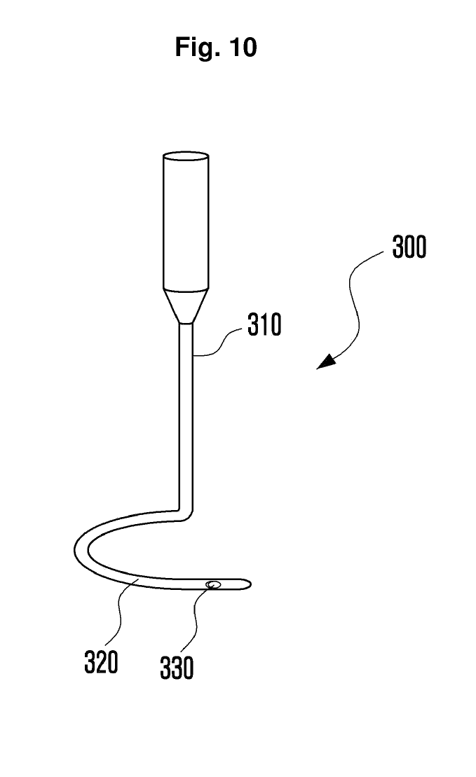

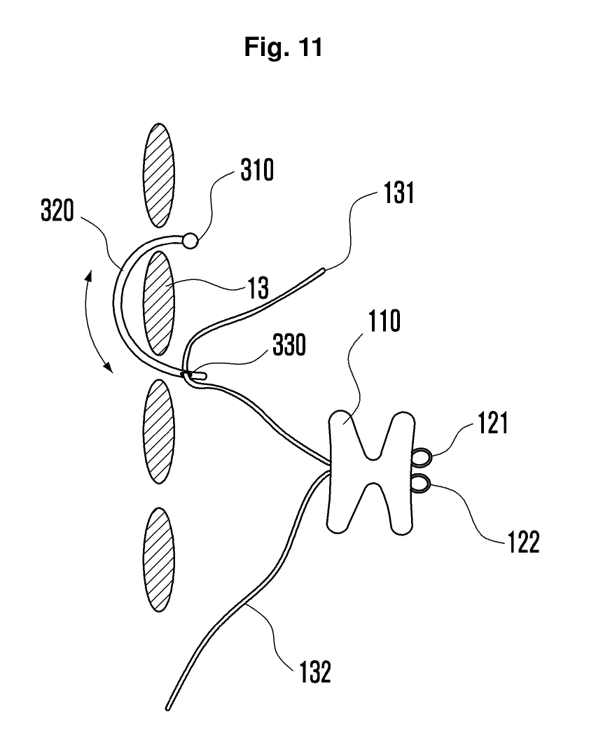

FIGS. 10 and 11 show another embodiment of a method for fixing an interspinous process support according to the present invention, in which needles provided at each end of the upper and lower straps 131, 132 are removed, and the straps 131, 132 pass around the spinous processes 13 using a strap drawer 300 as illustrated in FIG. 10.

To this end, the strap drawer 300 comprises a bar-shaped rotating handle 310, and a half-moon-shaped rotating needle 320 in a horizontal direction at the bottom of the rotating handle 310, and a strap rack hole 330 for hooking straps 131, 132 is provided at one end of the rotating needle 320.

Such a strap rack hole 330 may be of a ring shape or a loop shape.

Accordingly, when the rotating needle 320 of the strap drawer 300 is placed where the back muscles are peeled off and the rotating handle 310 is rotated, the rotating needle 320 is inserted between the two upper and lower spinous processes 13, and the end point of the rotating needle 320 passes around the upper spinous process 13.

Thereafter, when the upper strap 131 is hooked onto a strap rack hole 330 formed at the rotating needle 320 and the rotating handle 310 is rotated in the opposite direction, the rotating needle 320 is withdrawn to the originally inserted position while the rotating needle 320 draws the upper strap 131, and the upper strap 131 is pulled out in the opposite direction winding around the spinous processes 13.

The lower strap 132 may be wound around the lower spinous process 13 in the same manner, and the procedure afterwards is identical to the embodiment of the invention.

1. A support between spinous processes, comprising:
a main body comprising a support part having a size to be inserted between spinous processes of backbones and receiving parts accommodating spinous processes therein with protruded top and bottom sides of the support part;
two rack holes formed at one side of the main body; and
two straps attached to an other side of the main body.

2. The support of claim 1, wherein half-moon-shaped needles are provided at one end of the straps.

3. The support of claim 2, wherein a warp angle of the needle is over 180° following a circle.

4. A fixing method for an interspinous process support, comprising:
preparing an interspinous process support and a strap fixing pin;
peeling back muscles at only one side on spinous processes with respect to the spinous processes to be operated, then removing elements that have encroached an area inside a spinal canal by surgery;
inserting a needle of an upper strap attached to an interspinous process support from a peeled side of the back muscle between two upper and lower spinous processes, to pass around the upper spinous process and then pulling the end of the needle with a forceps;
inserting the needle of a lower part strap between the two upper and lower spinous processes, to pass around the lower part spinous process, and then pulling at the end of the needle with another forceps;
inserting an interspinous support between the two upper and lower spinous processes and to be safely received between the spinous processes;
passing each of upper and lower straps through upper and lower rings, respectively, inserting a strap fixing pin, and fixing the upper and lower straps each to the interspinous process support;
cutting a strap exposed outside the strap fixing pin; and
sealing the peeled parts.

5. A fixing method for an interspinous process support, comprising:
preparing an interspinous process support and a strap fixing pin of the invention;
peeling back muscles at only one side on spinous processes with respect to the spinous processes to be operated, then removing elements that have encroached an area inside a spinal canal by surgery;
inserting a rotating needle between two upper and lower spinous processes from where the back muscles are peeled off using a strap drawer which comprises a rotating handle of a bar shape and the rotating needle of a half-moon shape at a bottom of the rotating handle, and then rotating the rotating handle so that the an end point of the rotating needle passes around the upper spinous process;
hooking the upper strap onto a strap rack hole formed at the rotating needle passing around the spinous process and rotating the rotating handle in an opposite way, so that the rotating needle is withdrawn to an inserted position and the upper strap is pulled out of the spinous process;
inserting the rotating needle between two upper and lower spinous processes from where the back muscles are peeled off using a strap drawer, and then rotating the rotating handle so that the end point of the rotating needle passes around the lower part spinous process;
hooking the lower strap onto a strap rack hole formed at the rotating needle passing around the lower part spinous process and rotating the rotating handle in an opposite way, so that the rotating needle is withdrawn to an inserted position and the lower strap is pulled out of the lower spinous process;
inserting the interspinous support between two upper and lower spinous processes to be safely received between spinous processes;
passing each of upper and lower straps through upper and lower rings, respectively, and inserting a strap fixing pin and fixing upper and lower straps each to the interspinous process support;
cutting a strap exposed outside the strap fixing pin; and
sealing the peeled parts.
